(19) United States
(12) Patent Application Publication
(10) Pub. No.: US 2020/0164133 A1
(43) Pub. Date: May 28, 2020

(54) METHOD OF SENSING AND SENSING CANNULA FOR USE DURING CARDIAC SURGERY

(71) Applicants: Gilbert H. Johnson, Meridian, ID (US); Claude Brandeau, Hermosa Beach, CA (US)

(72) Inventors: Gilbert H. Johnson, Meridian, ID (US); Claude Brandeau, Hermosa Beach, CA (US)

(21) Appl. No.: 16/505,663

(22) Filed: Jul. 8, 2019

Related U.S. Application Data
(60) Provisional application No. 62/695,637, filed on Jul. 9, 2018.

(51) Int. Cl. A61M 1/36; A61M 25/10

(52) U.S. Cl. CPC A61M 1/3659; A61M 25/10; A61M 1/3609; A61M 2230/202; A61M 2230/50; A61M 2230/04; A61M 2230/205; A61M 2210/125

(57) ABSTRACT

The method and system of the present invention detects cellular electrical activity and/or temperature continuously in real time to indicate the level of cellular arrest in the myocardial and conductive cells, allowing for the adjustment of cardioplegia, temperature, and/or increasing or decreasing the ratio of blood to electrolytes (cardioplegia) in order to eliminate or minimize myocardial ischemia that occurs when cellular arrest is not obtained or maintained during cardiac surgery. In a preferred embodiment of the present invention, a conductive wire and/or a thermistor are imbedded in or on the walls of a retrograde and/or other cannula to detect low amplitude electrical activity and/or temperature, respectively. In an alternate preferred embodiment of the present invention sensors for electrolyte, pO₂, pCO₂, or cardiac enzymes could also be added or used in replacement of other sensors.

### CROSS-REFERENCE TO RELATED APPLICATION

This application claims the priority of provisional application Ser. No. 62/695,637 filed Jul. 9, 2018 (pending), the disclosure of which is hereby incorporated by reference herein.

### BACKGROUND OF THE INVENTION

The present invention relates to balloon cannulas or cannulae (the terms cannula or cannulae may be used interchangeably throughout) used for cardiovascular applications.

During cardiac surgery, a patient's heart usually is placed in visible myocardial arrest by delivering cardioplegia solution to significantly decrease the oxygen demands and myocardial ischemia of the patient's myocardial tissue. In the prior art, maximum cellular arrest or myocardial arrest usually is determined by reviewing the patient's electrocardiogram (ECG) and noting when it shows no electrical activity (i.e. flatline) such that no myocardial activity is directly observed.

In the prior art, cellular arrest may be obtained and managed by infusing an electrolyte-based solution (cardioplegia) or another suitable solution into the heart. The cardioplegia interferes with the electrical activity of the myocardium on a cellular level and causes cellular arrest. Another prior art method of managing the arrest is by controlling the temperature of the heart, whenever necessary.

When using cardioplegia to manage myocardial arrest, the cardioplegia is delivered to the heart through the use of either antegrade and/or retrograde cannula. Antegrade cannula may deliver cardioplegia in the normal direction of blood flow either through the aortic root cannula or coronary ostial cannula. In the prior art, for example, using an aortic root cannula, cardioplegia may be delivered at certain medically studied pressures into the cardiac arterial tree to cause cellular arrest of the patient's heart to prevent myocardial ischemia. In the prior art, for example, using a coronary ostial cannula, the cannula is held on or placed into the coronary ostia through which cardioplegia is infused into the cardiac arterial tree.

Alternatively, in the prior art, for example, when retrograde cannula are used, the retrograde cannula may infuse cardioplegia to the heart via the coronary sinus. Because prior art retrograde cannula infuse cardioplegia on the venous side (i.e. not in the direction of normal blood flow), these cannula may have either auto inflating or manual inflating balloons at their distal ends to temporarily block blood/cardioplegia from exiting the heart until the myocardial arrest is observed. In the prior art, depending on the equipment available or the doctor's preference, the balloons may be either manually inflated or automatically inflated as the cardioplegia solution flows through them. Cannulas with manual inflation require a clinician to inflate the balloon using a syringe in order to deliver cardioplegia to the myocardium. The balloons/cuffs on auto inflation cannula inflate as the cardioplegia flows through the cannula body.

In the prior art, cellular arrest is determined and maintained by observing the myocardial activity of the patient's heart through the use of an ECG (flatline) lack of any motion by the myocardium. Should cellular activity or myocardial ischemia be observed or suspected, reinfusion of cardioplegia may be done. Reinfusion may occur based upon either accepted time standards known in the art or, less favorably, by either observation of myocardial activity on the patient's ECG or by direct observation of a clinician. However, in the prior art, when the direct observation method is used to determine the reoccurrence of electrical activity, by the time it is observed, cellular protection may be lost. Alternatively, in the prior art, when using an ECG to determine the return of any cellular activity, while the ECG generally can record electrical signals in the range of 10 mm/mV (see, e.g., J Res Med Sci. 2011 June; 16(6): 750-755.), it has been shown (see, e.g., Ann Thorac Surg 41; 1986; pp. 372-377) that cellular activity may occur at levels, at least, to 10⁻⁶ V (which reflects microfibrillation), which is considerably at a lower range than what is presently capable of being detected and recorded by prior art ECG's. And although cardioplegia may cause cellular arrest at this level, the ability to accurately observe, measure and maintain this level of cellular arrest is presently uncertain by standard prior art methods and devices.

Specifically, low-amplitude electrical activity (i.e., microfibrillation) still can occur at levels that may not be observed via ECG and/or direct visualization methods. Persistent failure to detect this low-amplitude electrical activity in a patient during cardiac surgery may cause cellular ischemia or other injury, or cellular death of the patient's myocardial or conductive tissue. Thus, it would be beneficial in the art to be able to detect low-amplitude electrical activity in a patient's heart during invasive cardiac surgery so as to better protect the myocardial and conductive tissue. Also, it would be beneficial to ensure in real time that a sufficient amount of cardioplegic solution has been delivered to the patient's cardiac tissue during an invasive cardiac procedure so that the heart may remain in arrest to protect the myocardial and conductive tissue.

### SUMMARY OF THE INVENTION

A preferred method and system of the present invention detects low-amplitude electrical activity (LEA) generated by a patient's myocardial or conductive tissue during cardiovascular procedures in real-time. By being able to detect LEA, the amount and timing of cardioplegia during the procedure may be managed so as not to damage the fragile myocardial and cellular tissue of the patient's heart. In an alternative method and system of the present invention, the temperature generated by a patient's myocardial or conductive tissue during cardiovascular procedures may be detected in real time, which also may be used to manage so as not to damage the fragile myocardial and cellular tissue of the patient's heart during that procedure.

In a preferred embodiment of the present invention, a bipolar or unipolar sensing device having a first end and a second end is embedded in or on the body of a cannula having a corresponding first and second end. The second end of the cannula which contains the second end of the sending device is placed within the patient's heart or mediastinal area so that it may detect and transfer low-amplitude electrical activity (LEA) down to, at least, 10⁻⁶ V, in real time. In a preferred embodiment, the sensing device comprises a wire linked to a simple electrical circuit comprised of an anode and cathode capable of detecting LEA activity to monitor the cellular electrical activity so that as cellular electrical activity is detected by the sensing device, additional cardioplegia may be administered to maintain cellular arrest. In a preferred embodiment of the present invention, a thermistor may also be embedded in or on the cannula body. The thermistor is then placed into the heart or the mediastinal area where it is able to detect myocardial temperatures in the range of 0-40° C. In a preferred embodiment, the temperatures are displayed and/or recorded on a monitor so that when temperatures are either higher or lower than the optimal range to maintain cellular arrest, additional cardioplegia and/or heat or cold may be administered to the heart to maintain the patient's cellular arrest.

### BRIEF DESCRIPTION OF THE DRAWING

The objects and advantages of the invention will become apparent from the following detailed description of preferred embodiments thereof in connection with the accompanying drawings in which like numerals refer to like parts and in which:

FIG. 1 is a cross section of an embodiment of a retrograde cannula in accordance with the present invention.

FIG. 2 is a cross section of the retrograde cannula shown in FIG. 1 taken along lines 2-2 of FIG. 1.

FIG. 3 is a photograph of a preferred embodiment of the device of the present invention.

### DETAILED DESCRIPTION OF THE INVENTION

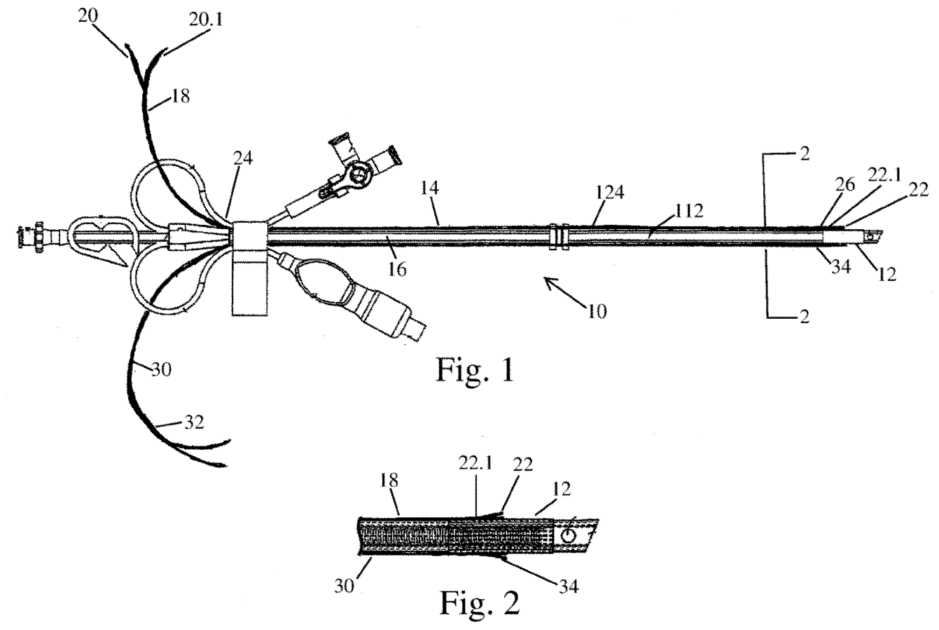

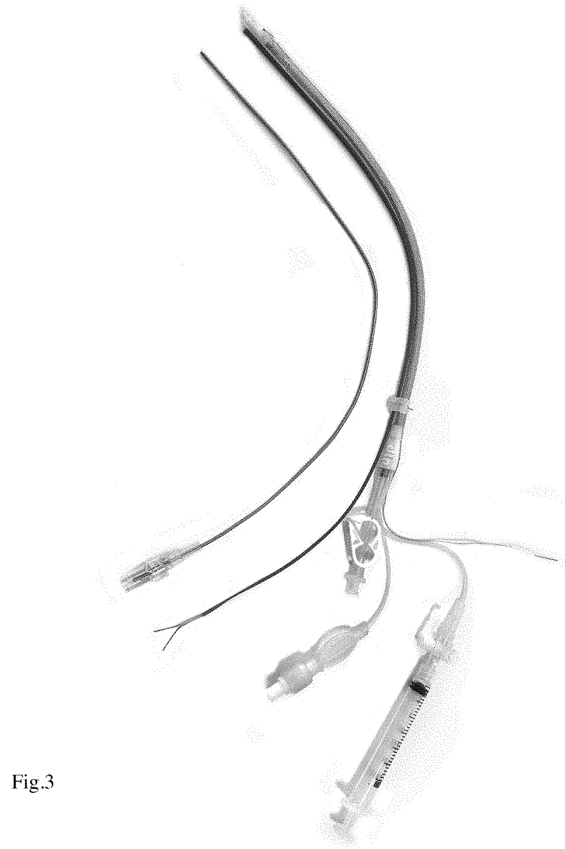

Referring to the drawings in which like numerals refer to like parts, referring first to FIGS. 1 and 3, the cross section of a cannula 10 according to the present invention is shown. Cannula 10 comprises a proximal end 24 and a distal end 26 which are attached through the cannula body 14 which, in a preferred embodiment, is comprised of a flexible medical grade plastic material such as PVC, silicone or urethane, although any flexible medical grade material known in the art may be used. Extending through the middle of cannula body 14 is a lumen 16 which is capable of conducting a fluid such as cardioplegic solution from the proximal end 24 to the distal end 26 of the cannula 10. Also embedded within or extending along the cannula body 14 or within the lumen 16 is a LEA sensor 18 having a proximal end 20 and a distal end 22 corresponding to the proximate end 24 and distal end 26 of the cannula 10. In a preferred embodiment of the present invention, LEA sensor 18 may comprise either a bipolar wire with the anode and cathode leads separated or a unipolar wire with the anode at the distal end of the cannula and the cathode place on an external normal thoracic landmark area. When a bipolar wire is used, the proximal end 20 and 20.1 of sensor 18 are connected to a 2-lead cable (not shown) comprised of an anode (not shown) and a cathode (not shown). Regardless of which kind of wire is used, the LEA sensor 18 monitors, displays and may also record the myocardial/electrical activity of the patient's heart.

In a preferred embodiment of the present invention, the LEA sensor 18 of the present invention senses extremely low amplitude electrical (LEA) signals in real time. In a preferred embodiment of the present invention, the LEA signals may be detected down to 10⁻⁶ V in real time, although even lower LEA signals may also be detected if greater sensitivity is required.

In a preferred embodiment of the present invention, such as the embodiment shown, a retrograde cannula is used, such that the distal end 26 has a manually inflatable balloon 12. An automatically inflating balloon cannula may be used instead, depending on the preference of the surgical team or the facility at which the surgery is being performed. Since the cannula 10 referred to the FIGURES is a retrograde cannula, when it is used, it is placed into the venous system via the patient's coronary sinus during surgery.

Using a preferred method of the present invention, when the distal end 26 of the cannula 10 is placed in position within the coronary sinus during surgery, should there be any cellular or myocardial activity, the sensor 18 will detect LEA signals in real time to notify the clinician/doctor that the cardiac cells may not be in full arrest. By being able to detect LEA in real time, cardiac arrest may be maintained such as by controlling the amount and timing of cardioplegia during the procedure so as not to damage the fragile myocardial and cellular tissue of the patient's heart.

In an alternative method and system of the present invention, the temperature generated by the blood/fluids that drain through the venous system into the right atrium and/or sensing the temperature of a patient's myocardial or conductive tissue during cardiovascular procedures also may be detected in real time, which also may be used to adjust cardioplegia temperature during that procedure to better control cardiac arrest. Specifically, in an alternate preferred embodiment, a thermistor 30 is embedded within or on the cannula body 14 or the lumen 16 which can monitor the temperature deep within the structure of the heart during the cardiac procedure. The thermistor has a proximal end 32 and a distal end 34. In a preferred method, monitoring the temperature of the heart permits optimal temperature management of cardioplegic solution which assists in maintaining the viability of myocardial and conductive cells. In a preferred embodiment, the thermistor 30 could be made of a flexible film. In another preferred embodiment, the thermistor could also be a fine wire.

In an alternate preferred embodiment of the present invention, both the sensor 18 and the thermistor 30 are used to provide optimal monitoring of the myocardial and conductive cellular activity.

In a preferred embodiment of the present invention, the receiver for the electrical and/or temperature signals may be a precision multimeter, such as Fluke 8845A or any other medical grade multimeter monitor with appropriate filters that can detect LEA signals and temperature.

In alternate embodiments of the present invention, other types of cannula may be used (not shown) such as antegrade cannula or venous cannula with sensing device 18 and/or thermistor 30 to provide optimal monitoring of the myocardial and conductive cellular activity in a similar fashion as described in the above retrograde cannula description.

Using the device and method of the present invention provides for the adjustment of cardioplegia, temperature, and/or increasing or decreasing the ratio of blood to electrolytes (cardioplegia) in order to eliminate or minimize myocardial ischemia that occurs when cellular arrest is not obtained or maintained during cardiac surgery.

In yet another alternate embodiment of the present invention, additional or alternate sensors (not shown) may be added, which is capable of sensing pO₂ or pCO₂.

As a further alternate embodiment of the present invention, sensors capable of detecting an electrolyte and/or a cardiac enzyme may be added or used alternatively to further assist in improving myocardial and conductive tissue function by increasing or decreasing the ratio of blood to electrolytes.

While particular embodiments and techniques of the present invention have been disclosed and illustrated herein, it will be understood that many variations, alternatives, substitutions, modifications and equivalents may be made by those persons skilled in the art without departing from the scope of the invention. It will be appreciated from the above description of presently preferred embodiments and methods that other configurations and techniques are possible and within the scope of the present invention. Thus, the present invention is not intended to be limited to the particular embodiments and techniques specifically discussed herein.

What is claimed is:

1. A cannula used that is placed within the heart during cardiac surgery, comprising:
a medical grade tubular body having a proximal and a distal end, the distal end adapted to be inserted into the heart;
a conductive wire for the purpose of sensing and conducting an electrical signal contained within or on the tubular body extending from the proximal end to the distal end, and
a low amplitude electrical signal detector connected to the proximal end of the conductive wire for detecting low amplitude signals occurring at a cellular level within the heart during cardiac surgery, whereby when cellular activity is detected by the signal detector, the ratio of blood to electrolytes (cardioplegia) is either increased or decreased in the heart in order to eliminate or minimize cellular ischemia that occurs when cellular arrest is not obtained or maintained during cardiac surgery.

2. The cannula of claim 1 further comprising a temperature sensor contained within or on the tubular body from the proximal end and the distal end for detecting the temperature levels of blood/fluids that drain through the venous system into the right atrium and/or sensing local myocardial tissue temperature in the heart during cardiac surgery.

3. The cannula of claim 1 wherein the signal detector and electrical sensor are capable of detecting low amplitude electrical signals down to 10⁻⁶ V in real time, although even lower LEA signals may also be detected if greater sensitivity is required.

4. The cannula of claim 1, further comprising a display connected to the signal detector for displaying the electrical signal detected by the detector.

5. The cannula of claim 2 wherein the signal detector may be a precision multimeter with appropriate filters that can detect LEA signals and temperature.

6. The cannula of claim 2, further comprising a display connected to the signal detector and the temperature sensor for displaying the electrical signal and temperatures detected by the signal detector and the temperature sensor.

7. The cannula of claim 2, wherein the temperature sensor is able to detect myocardial temperatures in the range of 0-40° C.

8. The cannula of claim 2, whereby when the detected temperature is not optimal to maintain cellular arrest, the temperature of the heart may be adjusted by administering either heat or cold and/or increasing or decreasing the ratio of blood to electrolytes (cardioplegia) in order to eliminate or minimize cellular ischemia that occurs when cellular arrest is not obtained or maintained during cardiac surgery.

9. A method for monitoring levels of cellular arrest during cardiac surgery and other cardiac procedures where cellular arrest is required, comprising,
sensing low amplitude electrical activity in real time down to 10⁻⁶ V in real time occurring within myocardial and conductive tissue of the heart.

10. The method of claim 9 further comprising, sensing temperature levels of blood/fluids that drain through the venous system into the right atrium and/or sensing local myocardial tissue temperature to determine whether management of cellular arrest should be by temperature and/or cardioplegia control.

11. The method of claim 10 further comprising, when an electrical signal is detected and/or the detected temperature of the levels of blood/fluids that drain through the venous system into the right atrium and/or the local myocardial tissue temperature in the heart during cardiac surgery is not optimal to maintain cellular arrest, adjusting the temperature by administering either warm or cold fluid and/or increasing or decreasing the ratio of blood to electrolytes (cardioplegia) is in order to eliminate or minimize cellular ischemia that occurs when cellular arrest is not obtained or maintained during cardiac surgery; and when an electrical signal is detected via the conductive wire reflecting low level cellular activity, giving additional cardioplegia to maintain cardiac arrest.

12. The method of claim 9 wherein the real time sensing of low amplitude electrical activity is capable of detecting electrical activity lower than 10⁻⁶ V.

13. The method of claim 10, wherein the temperature levels sensed are in the range of at least 0° C.-40° C. and are sensed in real time.

14. A cannula used to deliver cardioplegia to the heart to maintain cardiac arrest within the heart during cardiac surgery, comprising:
a medical grade tubular body having a proximal and a distal end, the distal end adapted to be inserted into the heart through which cardioplegia is delivered to the heart;
a conductive wire for the purpose of sensing and conducting an electrical signal contained within or on the tubular body extending from the proximal end to the distal end, and
a low amplitude electrical signal detector connected to the proximal end of the conductive wire for detecting low amplitude signals occurring at a cellular level within the heart during cardiac surgery,
a temperature sensor contained within or on the tubular body extending from the proximal end to the distal end of the cannula for detecting the temperature of the patient's heart during cardiac surgery,
whereby when the detected temperature is not optimal to maintain cellular arrest, the temperature of the heart may be adjusted by administering either warm or cold fluid and/or increasing or decreasing the ratio of blood to electrolytes (cardioplegia) in order to eliminate or minimize cellular ischemia that occurs when cellular arrest is not obtained or maintained during cardiac surgery; and
when an electrical signal is detected via the conductive wire, it reflects low level cellular activity requiring the need for additional cardioplegia to maintain cardiac arrest.

15. The cannula of claim 14 wherein the signal detector and electrical sensor are capable of detecting low amplitude electrical signals down to 10⁻⁶ V in real time, although even lower LEA signals may also be detected if greater sensitivity is required.

16. The cannula of claim 14 wherein the temperature sensor is able to detect myocardial temperatures in the range of 0-40° C.

17. The cannula of claim 14 wherein the signal detector may be a precision multimeter with appropriate filters that can detect LEA signals and temperature.

18. The cannula of claim 14, further comprising a display connected to the signal detector and the temperature sensor for displaying the electrical signal and temperatures detected by the signal detector and the temperature sensor.
